(19) United States
(12) Patent Application Publication
(10) Pub. No.: US 2022/0323743 A1
(43) Pub. Date: Oct. 13, 2022

(54) BLOOD PUMP

(71) Applicant: Gino Morello, Leonia, NJ (US)

(72) Inventor: Gino Morello, Leonia, NJ (US)

(21) Appl. No.: 17/718,921

(22) Filed: Apr. 12, 2022

Related U.S. Application Data
(60) Provisional application No. 63/173,835, filed on Apr. 12, 2021.

(51) Int. Cl. A61M 60/562; A61M 60/279; A61M 60/178; A61M 60/451; A61M 60/837; A61M 60/857

(52) U.S. Cl. CPC A61M 60/562; A61M 60/279; A61M 60/178; A61M 60/451; A61M 60/837; A61M 60/857; A61M 2205/0266

(57) ABSTRACT

A self-contained peristaltic pump includes a flexible flow conduit with a plurality of circumferential and/or longitudinal shapechange elements distributed longitudinally and/or transversely along the longitudinal axis of the flow conduit. The activations of the shapechange elements result in the positive displacement of fluid in the anterograde direction (i.e. from the anterior end of the pump to the posterior end).

### CROSS-REFERENCE TO RELATED APPLICATION

This application claims the benefit of U.S. Provisional Application No. 63/173,835, filed Apr. 12, 2021, and titled “BLOOD PUMP,” the disclosure of which is hereby incorporated herein by reference.

### BACKGROUND

This disclosure relates generally to blood pump systems, and more particularly to implantable peristaltic blood pump systems.

Generally, blood pump systems are employed in either of two circumstances. First, a blood pump may completely replace a human heart that is not functioning properly, or second, a blood pump may boost blood circulation in patients whose heart is still functioning although pumping at an inadequate rate. For example, many commercially available blood pumps are miniaturized continuous axial-flow or centrifugal pumps designed to provide additional blood flow to patients who suffer from heart disease. For example, the device is typically attached between the apex of the left ventricle and the aorta in the case of a left ventricle assist device (LVAD).

### BRIEF DESCRIPTION OF THE DRAWINGS

FIG. 1 illustrates a blood pump system in accordance with aspects of the present disclosure.

FIG. 2 illustrates another arrangement of the blood pump system in accordance with further aspects of the present disclosure.

FIG. 3 is a perspective view of an example of a flow conduit suitable for the blood pump system shown in FIGS. 1 and 2.

FIG. 4 is a perspective view of another example of a flow conduit suitable for the blood pump system shown in FIGS. 1 and 2.

FIG. 5 is a perspective view of a further example of a flow conduit suitable for the blood pump system shown in FIGS. 1 and 2.

FIG. 6 is a perspective view of another example of a flow conduit suitable for the blood pump system shown in FIGS. 1 and 2.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

As noted previously, blood pump systems may be employed to completely replace a human heart that is not functioning properly, it may be configured to boost blood circulation in patients whose heart is still functioning although pumping at an inadequate rate. For example, many commercially available blood pumps are miniaturized continuous axial-flow or centrifugal pumps designed to provide additional blood flow to patients who suffer from heart disease.

Many known blood pump systems typically are controlled in an open loop fashion where a predetermined speed is set and the flow rate varies according to the pressure differential across the pump. The pump itself may be controlled in a closed loop fashion, wherein the actual pump speed is fed back to a motor controller that compares the actual speed to the desired predetermined speed and adjusts the pump accordingly. Other pumps may be controlled in a closed loop fashion, in which the pump speed is varied according to a monitored parameter of the patient, such as the patient's pulse or blood flow rate.

Whether the pump is operated in a closed loop or open loop fashion, it is desirable to monitor several pump operating parameters, such as voltage level, current level, pump speed, flow rate, and the like. Adding additional measurement devices to the pump system, however, can complicate the system and add to the power requirements for the system.

Many prior axial and centrifugal continuous flow pumps have been developed but have not been entirely satisfactory due to a variety of problems. For example, fluid dynamic forces in the pump may activate platelets, leading to formation of blood clots. High shear stresses in the pump may damage red blood cells. These problems can be exacerbated by the small size required for a blood pump to be implantable, which necessitates a high rotational speed in a rotary type pump. Further, some prior devices have required large external support equipment, resulting in little or no patient mobility.

Problems of prior pumps that have limited their clinical use to relatively short times include the following: (1) blood damage which may occur when blood comes into contact with rotor bearings, (2) bearing seizure resulting from the considerable thrust and torque loads, or from dried blood sticking on the bearing surfaces, (3) problems of blood damage (hemolysis) and blood clotting (thrombosis) caused by relative rotational movement of the components of the pump, (4) pump and control size and shape limitations necessary for implantation or convenient mobility, (5) weight limitations for implantation to avoid tearing of implant grafts due to inertia of sudden movement, (6) difficulty in coordinating and optimizing the many pump design parameters which may affect hemolysis, (7) high power consumption that requires a larger power supply, (8) motor inefficiency caused by a large air gap between motor windings and drive magnets, (9) heat dissipation from the device to the body, (10) complex Hall Effect sensors/electronics for rotary control, (11) the substantial desire for minimizing percutaneous (through the skin) insertions, including support lines and tubes, (12) large pump and related hose internal volume which may cause an initial shock when filled with saline solution while starting the pump, and (13) high cost effectively makes the device unavailable for many patients who could otherwise benefit from it.

Although significant efforts have been made to solve such problems associated with implantable blood pumps, there is still a great need for an improved pump that can be used for an extended period in a human as a ventricle assist device, and that is reliable, compact, relatively inexpensive, requires limited percutaneous insertions, and produces fewer blood clots and less blood damage.

A peristaltic pump is a type of positive displacement pump used for pumping a variety of fluids. Currently available peristaltic pumps include an external roller assembly to function. The fluid is contained within a flexible tube fitted inside a circular pump casing (though linear peristaltic pumps have been made). A rotor with a number of “rollers”, “shoes”, “wipers”, or “lobes” or the like attached to the external circumference of the rotor compresses the flexible tube. As the rotor turns, the part of the tube under compression is pinched closed (or “occludes”) thus forcing the fluid to be pumped to move through the tube. Additionally, as the tube opens to its natural state after the passing of the cam (“restitution” or “resilience”) fluid flow is induced to the pump. This process is called peristalsis and is used in biological systems such as the gastrointestinal tract. Typically, there will be two or more rollers, or wipers, occluding the tube, trapping between them a body of fluid. The body of fluid is then transported, at ambient pressure, toward the pump outlet. Peristaltic pumps may run continuously, or they may be indexed through partial revolutions to deliver smaller amounts of fluid.

Peristalsis is a radially symmetrical contraction and relaxation of muscles that propagates in a wave down a tube, in an anterograde direction. Peristalsis includes involuntary movements of the longitudinal and circular muscles, primarily in the digestive tract but occasionally in other hollow tubes of the body, that occur in progressive wavelike contractions. Peristaltic waves occur in the esophagus, stomach, and intestines. The waves can be short, local reflexes or long, continuous contractions that travel the whole length of the organ, depending upon their location and what initiates their action.

Some disclosed examples generally relate to a peristaltic blood pump that can be implanted into the chest of a human and can be used to assist or replace a human heart in pumping blood. A ventricular assist device, for example, includes a pump configured to pump blood of a patient. Disclosed embodiments include a peristaltic pump for use as implanted or implantable and extracorporeal blood pump systems. The lack of mechanical contact bearings in disclosed pumps enables longer life pump operation and less damage to working fluids such as blood.

Some disclosed peristaltic pumps may employ Shapechange Elements, which may include Smart materials. Smart materials, also called intelligent or responsive materials, are designed materials that have one or more properties that can be changed in a controlled fashion by external stimuli, such as stress, moisture, electric or magnetic fields, light, temperature, pH, or chemical compounds. This change is reversible and can be repeated many times. There is a wide range of different smart materials. Each offer different properties that can be changed. Smart materials are the basis of applications such as sensors and actuators, or artificial muscles, particularly as electroactive polymers (EAPs). Terms used to describe smart materials include shape memory material (SMM) and shape memory technology (SMT).

Artificial muscles, also known as muscle-like actuators, are materials or devices that mimic natural muscle and can change their stiffness, reversibly contract, expand, or rotate within one component due to an external stimulus (such as voltage, current, pressure or temperature). The three basic actuation responses— contraction, expansion, and rotation can be combined together within a single component to produce other types of motions (e.g. bending, by contracting one side of the material while expanding the other side). Conventional motors and pneumatic linear or rotary actuators do not generally qualify as artificial muscles, because there is more than one component involved in the actuation.

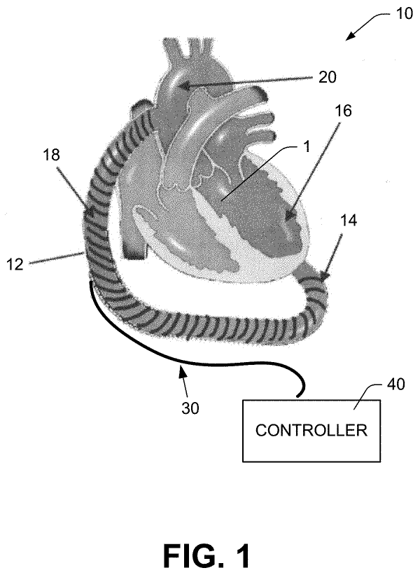

FIG. 1 shows an exemplary heart pump system 10 which as shown, functions as a ventricle assist device such as a left ventricle assist device (LVAD). The system 10 includes in implantable pump 12 (or “VAD pump”). As will be discussed further below, the pump 12 is a peristaltic pump that uses shapechange elements, which may include smart materials.

The pump 12 may be implanted trans-arterially/vascularly. In the example of FIG. 1, the pump 12 includes an inlet end 14 configured to connect to a first portion of a human heart 1. FIG. 1 illustrates an LVAD configuration and as such, the inlet end 14 connects to the left ventricle 16. An outlet or outflow end 18 connects to a second portion of the heart 1, such as the ascending aorta 20. The inlet 14 and outlet 18 ends may connect to the heart 1 in any suitable manner, such as by cannula(s). In other examples, the inlet 14 and/or outlet 18 are anastomosed directly to the heart (e.g. aorta and ventricle) with no cannulation.

A percutaneous cable 30 may be provided to connect the pump 12 or components thereof to an external power source and/or controller 40, for example, to provide control signals to the pump 12. In other embodiments wireless control configurations may be employed using suitable wireless communication schemes.

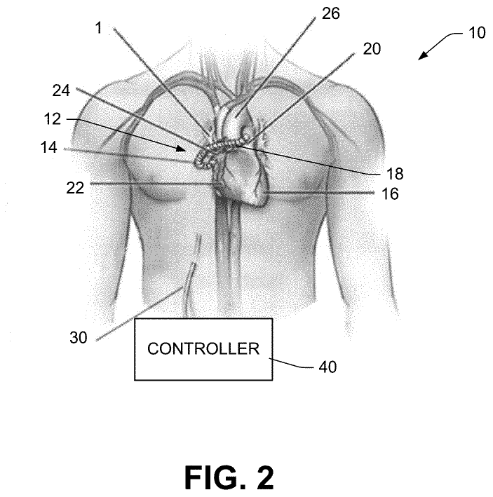

FIG. 2 illustrates a right ventral assist device (RVAD) configuration, in which the pump 12 is configured to pump blood from the inlet end 14 connected to the right ventricle 22 or right atrium 24 to the pulmonary artery 26. In the illustrated example, outlet end 18 connects to the aorta 20.

The pump 12 may be grafted within an existing vessel or placed circumferentially about an existing vessel (e.g. descending aorta). As noted above, disclosed examples of the pump 12 employ shapechange elements that may be configured and programmed such that the pump 12 functions as a left ventricular assist device or a right ventricular assist device. In other examples, dual pumps 12 may be configured and programmed to function as a biventricular support system, or dual pumps may be configured and programmed to function as a total artificial heart (e.g. both ventricles removed and atria preserved for attachment). The pumps may be anastomosed directly to the aorta and ventricle with no cannulation needed.

One disclosed pump 12 may be placed one inside another pump 12 to yield 100% redundancy and greater safety. Pumps 12 may be connected serially one to another to yield 100% redundancy and greater safety.

The peristaltic pump 12 allows pumping fluid, such as blood, without exposing the fluids to contamination from exposed pump components. In addition to pumping blood, the pump 12 may be configured for other pumping applications, including pumping IV fluids through an infusion device, apheresis, aggressive chemicals, high solids slurries and other materials where isolation of the product from the environment, and the environment from the product, are critical. It may also be used in heart-lung machines to circulate blood during a bypass surgery, and in hemodialysis systems, as the pump does not cause significant hemolysis.

The ideal peristaltic pump should have an infinite diameter of the pump head and the largest possible diameter of the rollers. Such an ideal peristaltic pump would offer the longest possible tubing lifetime and provide a constant and pulsation-free flow rate. Peristaltic pumps can be designed to approach these ideal peristaltic pump parameters. One example of a possible construction is depicted in the appended drawings. Disclosed examples offer constant accurate flow rates for extended periods together with a long tubing lifetime without the risk of tubing rupture.

With disclosed examples, the pumped fluid contacts only the inside surface of the pump tubing thereby negating concern for other valves, O-rings or seals that might be incompatible with fluid being pumped. Therefore, only the composition of the tubing that the pumped medium travels through is considered for chemical compatibility.

Elastomeric material may be employed to maintain the circular cross section after millions of cycles of squeezing in the pump. Example elastomers for pump tubing include nitrile (NBR), Hypalon, Viton, silicone, PVC, EPDM, EPDM+polypropylene (as in Santoprene), polyurethane and natural rubber. Of these materials, natural rubber has the best fatigue resistance, and EPDM and Hypalon have the best chemical compatibility. Silicone is popular with water-based fluids, such as in bio-pharma industry, but have limited range of chemical compatibility in other industries.

Extruded fluoropolymer tubes such as FKM (Viton, Fluorel, etc.) have good compatibility with acids, hydrocarbons, and petroleum fuels, but have insufficient fatigue resistance to achieve an effective tube life.

Some examples provide a broad chemical compatibility using lined tubing and fluoroelastomers. With lined tubing, the thin inside liner is made of a chemically resistant material such as poly-olefin and PTFE that form a barrier for the rest of the tubing wall from coming in contact with the pumped fluid. These liners are materials that are often not elastomeric, therefore the entire tube wall cannot be made with this material for peristaltic pump applications. This tubing provides adequate chemical compatibility and life to be used in chemically challenging applications. For those with need for chemically compatible tubing, these lined tubing offer a good solution.

With fluoroelastomer tubing, the elastomer itself has the chemical resistance. In the case of e.g. Chem-Sure, it is made of a perfluoroelastomer, that has the broadest chemical compatibility of all elastomers. The two fluoroelastomer tubes listed above combine the chemical compatibility with a very long tube life stemming from their reinforcement technology but come at a high initial cost. One has to justify the cost with the total value derived over the long tube life and compare with other options such as other tubing or even other pump technologies.

Advantages of peristaltic pumps include
No contamination. Because the only part of the pump in contact with the fluid being pumped is the interior of the tube, it is easy to sterilize and clean the inside surfaces of the pump.
They are able to handle slurries, viscous, shear-sensitive and aggressive fluids.
Pump design prevents backflow and siphoning without valves.
A fixed amount of fluid is pumped per rotation, so it can be used to roughly measure the amount of pumped fluid.

Some examples of smart materials suitable for use in the pump 12 include:
Piezoelectric materials that produce a voltage when stress is applied. Since this effect also applies in a reverse manner, a voltage across the sample will produce stress within sample. Suitably designed structures made from these materials can, therefore, be made that bend, expand or contract when a voltage is applied.
Photovoltaic materials or optoelectronics that convert light to electrical current.
Electroactive polymers (EAPs) that change their volume by voltage or electric fields.
Magnetostrictive materials that exhibit a change in shape under the influence of magnetic field and also exhibit a change in their magnetization under the influence of mechanical stress.
Magnetic shape memory alloys that change their shape in response to a significant change in the magnetic field.
Smart inorganic polymers showing tunable and responsive properties.
pH-sensitive polymers that change in volume when the pH of the surrounding medium changes.
Temperature-responsive polymers that undergo changes upon temperature.
Halochromic materials that change their color as a result of changing acidity.
Chromogenic systems that change color in response to electrical, optical or thermal changes. These include electrochromic materials, which change their color or opacity on the application of a voltage (e.g., liquid crystal displays), thermochromic materials change in color depending on their temperature, and photochromic materials, which change color in response to light—for example, light-sensitive sunglasses that darken when exposed to bright sunlight.
Ferrofluids, or other magnetic fluids (affected by magnets and magnetic fields).
Photomechanical materials that change shape under exposure to light.
Polycaprolactone (polymorph) materials that can be molded by immersion in hot water.
Self-healing materials that have the intrinsic ability to repair damage due to normal usage, thus expanding the material's lifetime.
Dielectric elastomers (DEs), which are smart material systems which produce large strains (up to 500%) under the influence of an external electric field.
Magnetocaloric materials that undergo a reversible change in temperature upon exposure to a changing magnetic field.
Smart self-healing coatings that heal without human intervention.
Thermoelectric materials that convert temperature differences into electricity and vice versa.
Chemoresponsive materials that change size or volume under the influence of external chemical or biological compound.
Shape-memory alloys and shape-memory polymers in which large deformation can be induced and recovered through temperature changes or stress changes (pseudoelasticity). The shape memory effect results due to respectively martensitic phase change and induced elasticity at higher temperatures.

Nitinol and Flexinol are two examples of shape-memory alloys often referred to as “Muscle Wires.” Muscle Wires are thin, highly processed strands of a nickel-titanium alloy called Nitinol—a type of Shape Memory Alloy that can assume radically different forms or “phases” at distinct temperatures.

At room temperature Muscle Wires are easily stretched by a small force. However, when conducting an electric current, the wire heats and changes to a much harder form that returns to the “unstretched” shape - the wire shortens in length with a usable amount of force. Some Muscle Wires can be stretched by up to eight percent of their length and will recover fully, but only for a few cycles. However when used in the three to five percent range, Muscle Wires can run for millions of cycles with very consistent and reliable performance.

Large wires are generally stronger than small ones, and strength varies with diameter. The strength to expect from a wire when heated is shown by the Recovery Weight in the table below. The Deformation Weight indicates the amount needed to stretch a wire when cool—about one sixth the force exerted when the wire is heated.

For more strength, two or more wires may be used in parallel. This provides as much strength as needed, and still keeps the fast cycle times of smaller wires. Muscle Wires contract as fast as they are heated - in one thousandth of a second or less. To relax, the wire must be cooled, which depends on the conditions surrounding the wire, and its size. For example, Flexinol HT series of wires has a higher transition temperature and cools up to 50% faster than the LT wires. The table below gives typical cycle rates for both LT and HT wires in still air. Moving air or immersing the wires in a fluid like a water/glycerin mixture can increase these by ten times or more.

| | Wire | Linear | Typical | Deform. | Recovery | Typical |
|---|---|---|---|---|---|---|
| | Diameter | Resistance | Current | Weight** | Weight** | Rate** |
| Wire Name | (microns) | (ohm/m) | (mA) | (grams) | (grams) | (LT/HT) |
| Flexinol 025 | 025 | 1770 | 20 | 2 | 7 | 55/na |
| Flexinol 037 | 037 | 860 | 30 | 4 | 17 | 52/68 |
| Flexinol 050 | 050 | 510 | 50 | 8 | 35 | 46/67 |
| Flexinol 075 | 075 | 200 | 100 | 16 | 80 | |
| Flexinol 100 | 100 | 150 | 180 | 28 | 150 | 33/50 |
| Flexinol 125 | 125 | 70 | 250 | 45 | 230 | |
| Flexinol 150 | 150 | 50 | 400 | 62 | 330 | 20/30 |
| Flexinol 200 | 200 | 32 | 610 | 116 | 590 | |
| Flexinol 250 | 250 | 20 | 1000 | 172 | 930 | 9/13 |
| Flexinol 300 | 300 | 13 | 1750 | 245 | 1250 | 7/9 |
| Flexinol 375 | 375 | 8 | 2750 | 393 | 2000 | 4/5 |

*Multiply by 0.0098 to get force in Newtons
**Cycles per minute, in still air, at 20 Centigrade
LT = low temp 70° C., HT high temp 90° C.

Compared to motors or solenoids, Muscle Wires have many advantages: small size, light weight, low power, a very high strength-to-weight ratio, precise control, AC or DC activation, low magnetism, long life, and direct linear action. To extend the lifetime and increase performance of Muscle Wires, good electrical and mechanical connections are provided, and the wire is protected overheating and overstraining. The power needed to activate a wire depends on its diameter, length, and the surrounding conditions. The table below gives typical current levels for “room temperature” conditions. Power can be increased, but once the wire has fully shortened, power should be reduced to prevent overheating.

Some disclosed embodiments of the pump 12 combine a flexible tubular flow conduit 100 with a plurality of electrically activated circumferential and/or longitudinal shapechange elements distributed longitudinally and/or transversely along the longitudinal axis of the flow conduit. The flexible flow conduit 100 may be tubular with a circular or non-circular cross-section in shape.

The shapechange elements may be placed external or internal to the flow conduit 100. Further, the shapechange elements may be placed within inner and outer walls of the flow conduit. Still further, the shapechange elements may be placed both on an inner and outer wall of the flow conduit for redundancy and for greater contractility. In other implementations, a monolithic single-element deformable shapechange conduit is configured to contract in a prescribed and deterministic manner based on the application location of stimuli (eg voltage, current, optical, acoustic signal) about its volume, rather than configuring separate shapechange element(s) relative to a flow conduit.

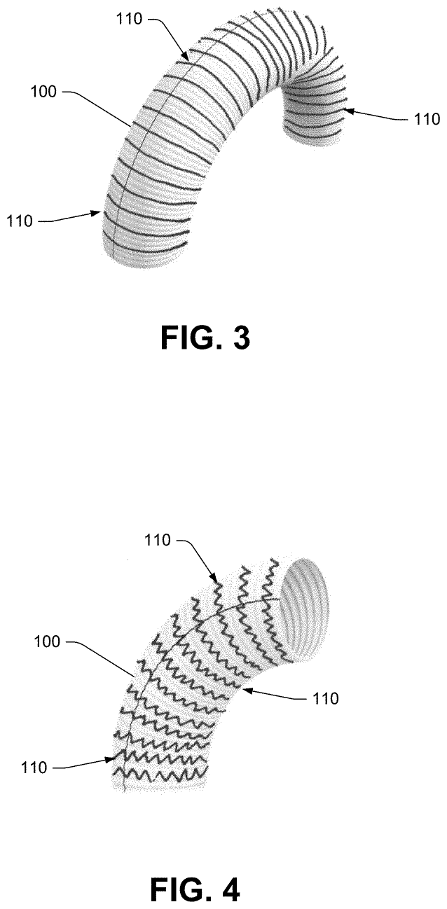

FIG. 3 and FIG. 4 illustrate example flexible tubular flow conduits 100 with a plurality of electrically activated circumferential shapechange elements 110 distributed transversely along the longitudinal axis of the flow conduit 100 in a spaced-apart relationship. In other examples, the shapechange elements 110 may are situated helically around and along the flexible flow conduit 100 at a given pitch along its longitudinal axis. The shapechange elements 110 shown in FIG. 3 and FIG. 4 have different geometric patterns. Such predetermined patterns may be selected to magnify the effects of the shapechange element shape (length) and tensile force.

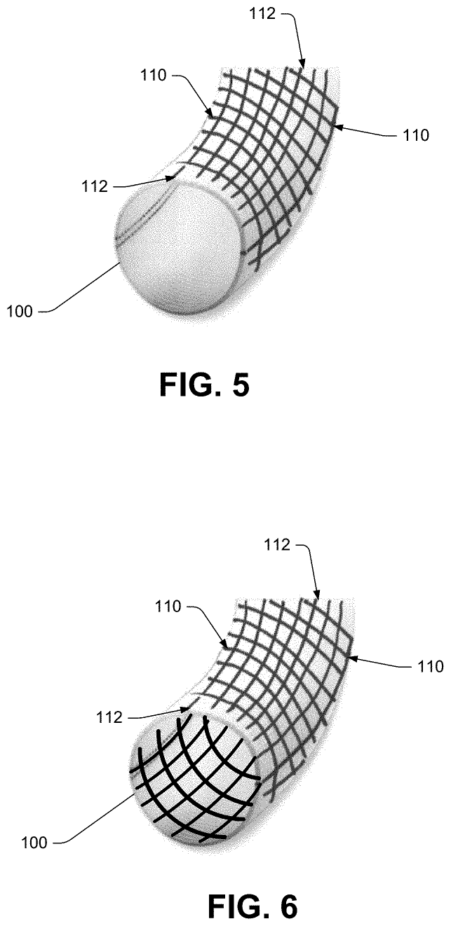

FIG. 5 illustrates another example of the flow conduit 100 having shapechange elements 112 configured longitudinally along the flexible flow conduit spaced radially (angularly) along the longitudinal axis in addition to the circumferential shapechange elements 110 distributed transversely along the longitudinal axis of the flow conduit 100. FIG. 6 illustrates yet another example of the flow conduit 100 having shapechange elements 112 configured longitudinally along the flexible flow conduit spaced radially (angularly) along the longitudinal axis in addition to the circumferential shapechange elements 110 distributed transversely along the longitudinal axis of the flow conduit 100 both internal and external thereto. In other embodiments, the shapechange elements 112 may be situated between inner and outer walls of the flow conduit 100.

Some embodiments may employ shapechange elements 110 made of a material whose shape changes with the application of an electrical signal (e.g. voltage, current). The shapechange elements 110 may be made of highly processed strands of a nickel-titanium shape memory alloy (e.g. Nitinol or Flexinol), a modified polymer, a material whose shape changes with the application of an optical signal, and/or a material whose shape changes with the application of an acoustic signal.

The shapechange elements may be made of highly processed strands of a nickel-titanium shape memory alloy (e.g. Nitinol or Flexinol), a modified polymer. One or more types of shapechange materials to may be employed optimize performance and safety.

Moreover, the plurality of shapechange elements 110 may be configured such that greater pumping action (e.g. contractility) is obtained through mechanical advantage obtained through the geometry, placement, and orientation of the shapechange elements 110. The pump 12 may be fabricated with shapechange elements 110 distributed over the flow conduit's 100 entire length or placed selectively to further control or optimize device performance. The pump 12 may be used with a protective semi-rigid sheath to prevent kinking (i.e. anti-kinking device). In some examples, the pump may be made with a radio opaque marker within the flexible flow conduit of the pump such that it may be visualized using x-ray, fluoroscope, and other safe and allowable imaging modalities for kink detection and placement.

The shapechange elements 110 may be configured, actuated, and modulated by the controller 40 such that the pump's 12 volumetric flow may be estimated from intrinsic pump signals (e.g. voltage, current, and frequency) by the controller 40 or another device receiving such pump signals. Further, the shapechange elements 110 may be configured, actuated, and modulated in a pattern to optimize blood flow and pulsation. Moreover, the shapechange elements 110 may be configured, actuated, and modulated such that the pump is intrinsically pulsatile which mimics the pumping action of the native human heart and may preclude the need for an additional valve to prevent retrograde flow (backflow). In other examples, the shapechange elements 110 are configured and actuated such that one group of the shapechange elements 110 are used to provide pumping action while an independent element or group of elements 110 may be used to occlude the pump 12 to ensure non-retrograde flow in the event of a pump 12 or primary driver failure. Further, the shapechange elements 110 may be configured, actuated, and modulated to optimize flow and protect the pulmonary side.

The shapechange elements 110 may utilize blood flow through the flow conduit 100 to cool down and increase rate of expansion of the shapechange elements 110. In some examples the shapechange elements 110 are configured and actuated by the controller 40 such that the pump 12 pumps in the anterograde direction (i.e. from the anterior end of the pump to the posterior end) only to preclude retrograde flow for safety.

The plurality of shapechange elements 110 may be configured, actuated, and modulated such that the pump 12 provides undulating pumping action, and/or sequential pumping action. The shapechange elements 110 may further be configured, actuated, and modulated such that the pump 12 provides custom fully programmable pumping action. The pump 12 may be made in various diameters based on application, pump requirements, and site usage. The pulsation mechanism scales linearly accordingly.

The shapechange elements 110 may be modulated via proportional or on/off control (e.g. by the controller 40). For instance, the shapechange elements 110 may be modulated via proportional or on/off control to generate a desired heart rate, ejection fraction, pressure waveform, and related physiologic performance. Further, the shapechange elements 110 may be modulated via proportional or on/off control to generate a gradual reduction of percentage ejection to help recover the native heart.

The shapechange elements 110 may be configured for valve-less or valved operation, and may be independently configured, actuated, and modulated such that an individual element or element driver failure does not preclude the remaining elements from generating pumping action for safety.

The pump 12 may be implanted trans-arterially/vascularly. The pump 12 may be grafted within an existing vessel or placed circumferentially about an existing vessel (e.g. descending aorta). In other examples, dual pumps 12 may be configured and programmed to function as a biventricular support system, or dual pumps 12 may be configured and programmed to function as a total artificial heart (e.g. both ventricles removed and atria preserved for attachment). As noted above, the pump(s) 12 may be anastomosed directly to the aorta and ventricle with no cannulation needed.

Embodiments of the pump 12 are thus small, lightweight, inherently pulsatile, provide anterograde flow, require no bearings, do not need a valve to prevent back flow, and may be programmed through an electronic controller 40 to adjust pulse rate, volumetric output, and pressure.

The activations of the shapechange elements 110 result in the positive displacement of fluid in the anterograde direction (i.e. from the anterior end of the pump to the posterior end). The length and diameter of the flow conduit 100 define the basis for the displacement of the pump 12 while the actuation of the shapechange elements 110 define the basis for flow rate, pulsatility, and pressure of the pump. The electronic controller 40 is used to programmatically actuate the shapechange elements and optimized to minimize hemolysis while maintaining pump efficiency.

The amount of squeeze applied to the tubing 100 affects pumping performance and the tube life—more squeezing decreases the tubing life dramatically, while less squeezing can cause the pumped medium to slip back, especially in high pressure pumping, and decreases the efficiency of the pump dramatically and the high velocity of the slip back typically causes premature failure of the hose. Therefore, this amount of squeeze becomes an important design parameter.

The term “occlusion” is used to measure the amount of squeeze. It is either expressed as a percentage of twice the wall thickness, or as an absolute amount of the wall that is squeezed. For instance, let

y=occlusion

g=minimum gap between the roller and the housing

t=wall thickness of the tubing

Then

y=2t−g (when expressed as the absolute amount of squeeze)

y=(2t−g)/(2t)×100 (when expressed as a percentage of twice the wall thickness)

The occlusion is typically 10 to 20%, with a higher occlusion for a softer tube material and a lower occlusion for a harder tube material.

Thus for a given pump, a critical tubing dimension becomes the wall thickness. The inside diameter of the tubing may not be an important design parameter for the suitability of the tubing for the pump. Therefore, it is common for more than one ID be used with a pump, as long as the wall thickness remains the same.

For a given pulsation rate of the pump 12, a tube 100 with larger inside diameter (ID) will give higher flow rate than one with a smaller inside diameter. Intuitively the flow rate is a function of the cross-section area of the tube bore.

The flow rate in a peristaltic pump is determined by many factors, such as:

Tube ID—higher flow rate with larger ID

Pump head OD—higher flow rate with larger OD

Pump head RPM—higher flow rate with higher RPM

Inlet Pulsation—the pulse reduces the filling volume of the hose

Increasing the number of actuators does not necessarily increase the flow rate, and it may decrease the flow rate somewhat by reducing the effective (i.e. fluid-pumping) circumference of the head. Increasing actuators does tend to decrease the amplitude of the fluid pulsing at the outlet by increasing the frequency of the pulsed flow.

The length of tube (measured from initial pinch point near the inlet to the final release point near the outlet) does not affect the flow rate. However, a longer tube implies more pinch points between inlet and outlet, increasing the pressure that the pump can generate.

The flow rate of a peristaltic pump is in most cases not linear. The effect of pulsation at the inlet of the pump changes the filling degree of the peristaltic hose. With high inlet pulsation the peristaltic hose will become oval and this is resulting in less flow. Accurate metering with a peristaltic pump is therefore only possible when the pump has a constant flow rate, or when inlet pulsation is complete eliminated with the use of correct designed pulsation dampeners.

The pulsation in a peristaltic pump is determined by many factors, such as:

Flow Rate—higher flow rate is more pulsation

Line Length—Long pipe lines is more pulsation

Higher Pump Speed—higher RPM is more pulsation

Specific gravity of the fluid—higher fluid density is more pulsation

Disclosed embodiments of the pump 12 may utilize readily accepted and approved graft materials (e.g. Dacron). In general, as noted above, examples of the pump 12 do not use high-velocity rotor/impellers which can generate high sheer forces and damage the relatively large red blood cells (hemolysis). Conventional mechanical bearings and bearing supports are eliminated and therefore pump thrombus formation, potential occlusion, and potential pump failure are minimized.

Active magnetic bearings are also not required, and therefore power, heat dissipation, pump complexity are minimized while increasing reliability. No rotating elements and related bearings are used, allowing the pump to run silently. Metal components may be avoided in the pump construction, thus the disclosed pump weighs significantly less than typical blood pumps. Examples of the disclosed pumps are easy to sterilize and the interior of the flow conduit easy to keep free of contaminants.

Various modifications and alterations of this disclosure may become apparent to those skilled in the art without departing from the scope and spirit of this disclosure, and it should be understood that the scope of this disclosure is not to be unduly limited to the illustrative examples set forth herein.

What is claimed is:

1. A pump, comprising:
a flexible tubular flow conduit having an inlet and an outlet, the inlet configured to attach to a first portion of a heart, the outlet configured to attach to a second portion of a heart; and
an actuator including a shapechange element configured to selectively deform the flow conduit in response to a received actuation signal.

2. The pump of claim 1, further comprising a controller configured to output the actuation signal to the actuator.

3. The pump of claim 1, wherein the plurality of shapechange elements are situated external to the flow conduit.

4. The pump of claim 1, wherein the plurality of shapechange elements are situated internal to the flow conduit.

5. The pump of claim 1, wherein the plurality of shapechange elements are situated within an inner and outer wall of the flow conduit.

6. The pump of claim 1, wherein the plurality of shapechange elements include a plurality of types of shapechange materials.

7. The pump of claim 1, wherein the plurality of shapechange elements are configured circumferentially around the flow conduit and spaced along a longitudinal axis of the flow conduit.

8. The pump of claim 1, wherein the plurality of shapechange elements are configured longitudinally along the flow conduit and spaced radially along a longitudinal axis of the flow conduit.

9. The pump of claim 1, wherein the plurality of shapechange elements are configured helically around the flow conduit at a given pitch along a longitudinal axis of the flow conduit.

10. The pump of claim 1, wherein the plurality of shapechange elements are configured in a predetermined geometric pattern.

11. The pump of claim 2, the actuation signal is configured to acutate the plurality of shapechange elements in a pattern to optimize blood flow and pulsation.

12. The pump of claim 1, wherein the plurality of shapechange elements are made of a material whose shape changes with the application of at least one of an electrical signal and/or an optical signal and/or an acoustic signal.

13. The pump of claim 2, wherein the controller is configured to modulate the actuation signal via proportional and/or on/off control.

14. The pump of claim 1, wherein the plurality of shapechange elements are configured for valve-less or valved operation.

15. The pump of claim 1, wherein the plurality of shapechange elements are independently actuatable.

16. The pump of claim 1, wherein the plurality of shapechange elements includes a first group of the shapechange elements configured to provide pumping action and a second group of the shapechange elements configured to occlude the pump.

17. The pump of claim 1, wherein the pump configured as one of a left ventricular assist device or a right ventricular assist device.

18. The pumps of claim 1, wherein the first portion of the heart includes a ventricle and the inlet is configured to be anastomosed directly to the ventricle, and wherein the second portion of the heart includes an aorta and the outlet is configured to be anastomosed directly to the aorta.

19. The pump of claim 1, further comprising a plurality of flow conduits including the flow conduit, wherein the plurality of flow conduits are arranged one inside another.

20. The pump of claim 1, further comprising a plurality of flow conduits including the flow conduit, wherein the plurality of flow conduits are connected serially to one another.
